(19) United States
(12) Patent Application Publication
(10) Pub. No.: US 2026/0131910 A1
(43) Pub. Date: May 14, 2026

(54) Protective Suit

(71) Applicant: Rambhatla Enterprises Pty Ltd, Botany NSW (AU)

(72) Inventor: Kiriti Rambhatla, Botany NSW (AU)

(21) Appl. No.: 19/121,646

(22) PCT Filed: Oct. 16, 2023
(86) PCT No.: PCT/AU2023/051018

§ 371 Date: Apr. 16, 2025

(30) Foreign Application Priority Data
Oct. 16, 2022 (AU) 2022903030

(51) Int. Cl. B64G 6/00; A61B 5/00

(52) U.S. Cl. CPC B64G 6/00; A61B 5/6804; A61B 2562/164

(57) ABSTRACT

An embodiment provides a protective suit system that is configured for protecting an occupant in extreme environments. The system may comprise a base layer to be worn on the occupant that is configured for thermal regulation of the occupant, the base layer including a biomarker sensor network for monitoring one or more biomarkers of the occupant. The system may also comprise an outer layer to be worn over the base layer, the outer layer including an exoskeleton structure sized so that the occupant can fit within the exoskeleton structure, and an exoskeleton covering that covers and is secured to the exoskeleton structure. The system may also comprise a data storage system that can store data generated by the biomarker sensor network.

### FIELD

The present disclosure relates to a protective suit system used to protect an occupant in extreme environments, such as in outer space.

### BACKGROUND

Protective systems are used to enable humans to operate in increasingly hostile environments, such as those on land and in space for contested chemical, biological, radiological and nuclear (CBRN) threat environments. When an individual needs to operate in these CBRN environments, a protective suit is employed to protect the occupant.

The type of environment and the tasks that need to be performed in these environments can determine the type of suit. For physically demanding tasks, it is often the case that the ability of the occupant to perform these tasks is limited by the suit. For example, a suit that allows great occupant mobility tends to be large and cumbersome or may only provide limited protection. This is not ideal.

The requirements of space suits often present specialised problems. The environment in outer space requires a life support system, plus protection from extreme temperatures and radiation. These factors tend to result in a suit that is large and bulky, requiring excess energy expenditure by the occupant in use. This energy expenditure limits the use of these suits to shorter operations.

Further, to date, space suits have been custom-made to accommodate occupant requirements and are designed based on feedback from the occupant on the overall fit and feel. This process limits the mass production of space suits and produces space suits that are not always correctly fitted. As the number of people travelling to space and requiring space suits is set to dramatically increase over the next decade, developing space suits requires a new approach.

### SUMMARY

An embodiment provides a protective suit system that is configured to protect an occupant in extreme environments, the system comprising:
a base layer to be worn on the occupant that is configured for thermal regulation of the occupant, the base layer including a biomarker sensor network for monitoring one or more biomarkers of the occupant;
an outer layer to be worn over the base layer, the outer layer including an exoskeleton structure that is sized so that the occupant can fit within the exoskeleton structure, and an exoskeleton covering that covers and is secured to the exoskeleton structure;
a data storage system that can store data generated by the biomarker sensor network.
The biomarkers of the occupant may include musculoskeletal biomarkers. The biomarker sensor network may include one or more biomarker sensors placed proximal to or on locations that are associated with synovial joints, heart and/or forearm of the occupant. In-use, the one or more sensors may non-invasively monitor the biomarkers. The protective suit system may comprise a plurality of biomarker sensors that are distributed on the base layer so that the biomarker sensors monitor in-use one or more of the occupant's shoulders, elbows, knees, ankles, heart and forearms.

The protective suit system may further comprise a life support system. The life support system may comprise a temperature regulation system that is operable with the base layer to regulate a temperature of the occupant. The temperature regulation system may include a thermal regulation fluid, a heat exchanger in thermal communication with the thermal regulation fluid, and a pump for pumping the thermal regulation fluid. The base layer may include a network of fluid channels through which the thermal regulation fluid can pass for regulating a temperature of the occupant. The base layer may comprise a first layer and a second layer. The network of fluid channels may be positioned between the first layer and the second layer.

The life support system may comprise an air management system that in use manages an air environment located within the outer layer in which the occupant is located in use of the protective suit system. The air management system may be configured to maintain a level of oxygen and carbon dioxide in the air environment within a predefined condition. The life support system may comprise a vent for venting the air environment located within the outer layer. The life support system may comprise a battery pack for powering at least the life support system, and a battery management interface for managing the battery pack. The battery pack may be replaceable. The battery management interface may include physical and/or digital inputs and controls.

The base layer may be formed from a breathable material with four-way stretch properties. The biomarker sensor network may be wirelessly connected to an internet of things network. The internet of things network may store or rely on data in the data storage system. The base layer may include a compression layer that is configured to apply pressure to the occupant. The compression layer may be formed from a stretchable material that covers an outer surface of the base layer. The compression layer may cover an entirety of the outer surface of the base layer. The compression layer may be fixed to the base layer.

The exoskeleton covering may comprise a plurality of layers of fabric. The plurality of layers of fabric may include one or more fabrics that are flame-resistant, flame-retardant, ballistic-proof, self-healing and/or resistant to radiation. The exoskeleton covering may be configured to operate in a temperature ranging from −270° C. to 1,260° C. The exoskeleton covering may be flame retardant and ballistic-proof. The exoskeleton covering may have stretchable bellow-like formations or materials at locations proximate or at the occupant's shoulders, elbows, hips, knees and ankles of the occupant.

The exoskeleton structure may include a torso section that is pivotally connected to a leg section. The torso section may in-use extend from the shoulders down to the hips of the occupant. The leg section may in-use extend down from the hips. The torso section may include a flexible spinal column that branches into a wishbone at a lower section of the flexible spinal column. The wishbone may be curved such that it extends down and around to attach to opposed sides of the leg section. The flexible spinal column may also branch into a shoulder platform at an upper section of the spinal column. The shoulder platform may extend laterally so as to fit in-use over the back side of the shoulders of the occupant. The leg section may include a hip portion that extends around the hips and includes two leg holes that can receive respective legs of the occupant, and a limb portion extending down each outer side of the hip portion. Each limb portion may include an articulation portion located at a knee region of the occupant. The limb portion may include one or more circular leg guards that can receive a leg of the occupant.

The outer layer may include a biomechanics sensor network that is configured to record data related to movement of the outer layer to monitor occupant energy usage and wear of the outer layer. Data generated by the biomechanics sensor network may be received by and stored in the data storage system.

The protective suit system may further comprise a helmet that is connectable to the outer layer and the data storage system. The helmet may comprise a cognitive tracking sensor that can track the cognitive function of the occupant. The helmet may comprise a display that provides a means of communicating information associated with the protective suit system to the occupant including information from the biomarker sensor network and the data storage system.

The protective suit system may further comprise a boot. The boot may include a boot temperature sensor for measuring a temperature of a foot of the occupant received in the boot in use and/or a temperature of an environment outside of the boot. The boot may include a boot pressure sensor for measuring pressure being applied by the occupant and/or the protective suit down onto a surface on which the occupant is standing. Data generated by the boot temperature sensor and boot pressure sensor may be received by and stored in the data storage system.

In an embodiment, the protective suit system may be a space suit.

### BRIEF DESCRIPTION OF THE DRAWINGS

Embodiments will now be described, by way of example only, with reference to the accompanying non-limiting drawings, in which:

FIG. 1 is a perspective view of a base layer.

FIG. 2 is a perspective view of a base layer.

FIG. 3 is a schematic cross-sectional view of an embodiment of a base layer.

FIG. 4 is a schematic cross-sectional view of an embodiment of a base layer.

FIG. 5 is a schematic cross-sectional view of an embodiment of a base layer.

FIG. 6 is a schematic cross-sectional view of an embodiment of a base layer.

FIG. 7a shows a front perspective view of an exoskeleton structure fitted over an occupant who is wearing the base layer.

FIG. 7b is a rear perspective view of the exoskeleton structure in FIG. 7a.

FIG. 8 is a front view of an embodiment of an outer layer.

FIG. 9 is an embodiment of an architecture of a protective suit system.

### DETAILED DESCRIPTION

An embodiment relates to a protective suit system that is configured for protecting an occupant in extreme environments. In the following description, the protective suit system is embodied as a space suit. However, the disclosure is not limited to a space suit and the protective suit system could be used on land or in water for activities related to chemical, biological, radiological and nuclear (CBRN) environments, and could be used in real scenarios or for training or simulations.

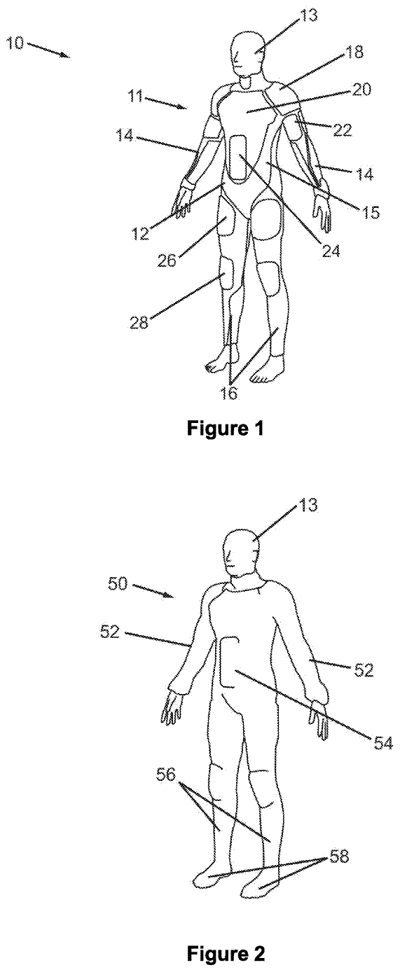

Now referring to FIG. 1, the protective suit system is in the form of space suit 10. Space suit 10 has a base layer in the form of sensor suit 11. The sensor suit 11 is worn by an occupant 13 and is sized to be tight fitting on the occupant 13. The sensor suit 11 includes a garment 12 that has arm portions 14, a torso portion 15, and leg portions 16. The sensor suit 11 functions include thermal regulation of the occupant and biomarker monitoring. Thermal regulation is achieved by incorporating a network of fluid channels into the sensor suit 11. In use, a temperature regulation fluid can be passed through the fluid channels to regulate a temperature of the occupant 13.

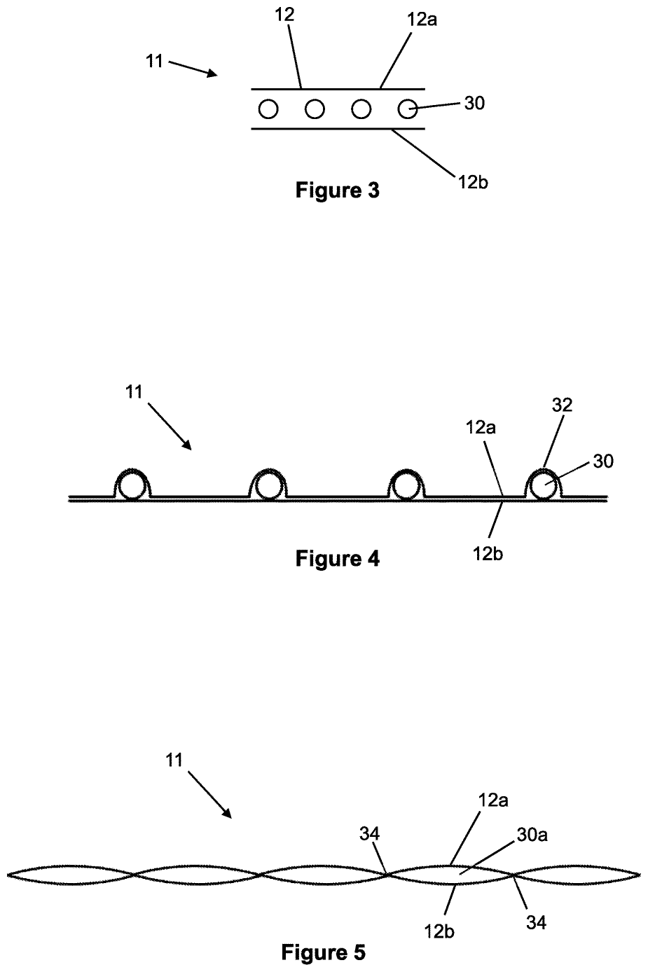

As shown in FIG. 3, the fluid channels can be in the form of tubes 30 that are arranged to form a pipe network that covers areas of the sensor suit 11. In an embodiment, the tubes 30 are positioned or sandwiched between a first garment layer 12a and a second garment layer 12b of the garment 12. In an embodiment, the tubes 30 are sewn into the garment 12 by sewing the first garment layer 12a and the second garment layer 12b together. In an embodiment, and as shown in FIG. 4, the first garment layer 12a and the second garment layer 12b are sewn together to form a hollow passage 32 within which the tubes 30 are positioned. In an embodiment, and as shown in FIG. 5, the first garment layer 12a and second garment layer 12b are joined together via contact or weld points 34 that define a hollow 30a through which a temperature regulation fluid can pass through. Alternatively, a tube may be passed through the hollow 30a (not shown). The weld points 34 are shown in FIG. 5 as being a small contact point, but the weld points 34 could be an extended region similar to the arrangement shown in FIG. 4. In an embodiment, the fluid channels e.g. tubes 30 are positioned on an outside surface of the garment 12. Whatever form the fluid channels take, they can circulate a thermal regulation fluid through the garment 12 to regulate a temperature of the occupant 13. In an embodiment, the tubes 30 are affixed to one side of the garment 12, such as an inner or outer side. This arrangement may be used in addition to or as an alternative to being positioned between garment layers.

In an embodiment, the pipe network is positioned to cover an entirety of the garment 12. In an embodiment, the pipe network covers select areas of the garment 12. For example, the pipe network may be positioned over areas of the occupant that have greatest effect on thermal regulation. The positioning of the pipe network and orientation of the tubes 30 in the pipe network may be arranged to maximise the flexibility and movement of the garment 12.

The sensor suit 11 also has a biomarker sensor network incorporated into the garment 12. The biomarker sensor network is configured to monitor one or more biomarkers of the occupant 13 during use of the space suit 10. The biomarker sensor network includes one or more biomarker sensors placed proximal to or on or at locations that are associated with synovial joints, heart and/or forearm of the occupant 13. For example, as shown in FIG. 1, the biomarker sensor network includes a shoulder sensor 18, an upper arm sensor 22, a chest and heart sensor 20, an abdominal sensor 24, an upper leg sensor 26, and a knee sensor 28. The biomarker sensor network may also include ankle sensors. In an embodiment, one or more of the sensors 18, 20, 22, 24, 26 and 28 non-invasively monitor the biomarkers of the occupant 13. For example, the sensors 18, 20, 22, 24, 26 and 28 may contact or be placed proximal to the skin of the occupant 13. The sensors 18, 20, 22, 24, 26 and 28 are shown in FIG. 1 as occupying different regions of the garment 12. One large sensor may occupy each region, or a plurality of sensors may work together to form each sensor region or sensor array. For example, shoulder sensor 18 may include individual sensors that monitor specific muscles of the shoulder such as the trapezius, pectoralis major, deltoid and upper regions of the long and short bicep.

In an embodiment, the biomarkers include musculoskeletal biomarkers. For example, monitoring musculoskeletal biomarkers can help to monitor the amount of energy the muscles of the occupant 13 are using, and where that energy is being used e.g. abdominal vs arms. The biomarkers can also include physiological biomarkers such as blood O₂ levels, blood pressure, and heart rate. The biomarkers may also include excretory products present in sweat.

The garment 12 may also include pressure sensors to monitor the pressure applied to the skin of the occupant 13. For example, when the occupant 13 moves and presses against an object, such as a component of an exoskeleton structure 102 (referred herein as “exoskeleton 102”), the pressure sensors can detect the pressure applied to the occupant 13 by the object. The pressure sensors may form part of the biomarker sensor network.

The sensors of the biomarker sensor network may be positioned on a skin-side of the garment 12. In addition to or alternatively, the sensors of the biomarker sensor network may be embedded within the garment 12, such as positioned between the first layer 12 and second layer 12b similar to the tubes 30. The biomarker sensors of the biomarker sensor network may also be positioned on an outside surface of the garment 12.

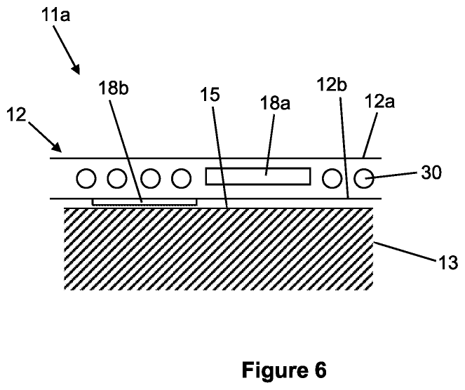

An embodiment of a sensor suit 11a is shown in FIG. 6, which shows an area associated with a shoulder of the occupant 13. The sensor suit 11a has a garment 12 where tubes 30 are positioned between the first layer 12a and the second layer 12b. A first shoulder sensor 18a is also positioned between the first layer 12a and the second layer 12b, while a second shoulder sensor 18b is positioned on a skin-side of the second layer 12b so that it directly contacts the skin 15 of the occupant 13 in use of the garment 12. The arrangement of the sensor suit 11a depicted in FIG. 6 is exemplary only and the relative positions of the sensors 18a and 18b and the tubes are merely to explain concepts of the current disclosure and can be applied to other sensors. One of the first shoulder sensor 18a and the second shoulder sensor 18b may be omitted from the sensor suit 11a.

To prevent restriction of movement of the occupant 13, the garment 12 is formed from a four-way stretch material. In an embodiment, the garment 12 is formed from or includes a breathable material to provide occupant 13 comfort in use of the garment 12.

In an embodiment, the sensor suit 11/11a includes a compression layer 50, which is best seen in FIG. 2. The compression layer 50 is formed from a stretchable material that has arm portions 52, a torso portion 54, leg portions 56 and feet portions 58, and is positioned over the garment 12. In an embodiment, the arm portions 52, the torso portion 54, leg portions 56, and feet portions 58 are integral. The compression layer 50 is configured to apply pressure to the occupant 13 to help counteract the effect of microgravity. As shown in FIG. 2, in an embodiment the compression layer 50 covers an entirety of an outer surface of the sensor suit 11. However, the compression layer 50 may instead only cover a portion of the garment 12 (not shown). In an embodiment, the compression layer 50 is secured or fixed to the garment 12, for example using adhesives, by welding and/or sewing. The compression layer 50 may be integral with the garment 12. Having the compression layer 50 be integral or fixed to the garment 12 may make it easier for the occupant to put on the compression layer 50. An inside of the compression layer 50 and/or the sensor suit 11/11a may be provided with a reduced friction coating to assist with the occupant 13 putting on and taking off the compression layer 50 and/or sensor suit 11/11a. In uses where effects of microgravity do not need to be considered, the compression layer 50 may not be required. Accordingly, the compression layer is not needed in all embodiments. In an embodiment, the compression layer 50 is a separate item to the sensor suit 11 and is worn over the sensor suit 11.

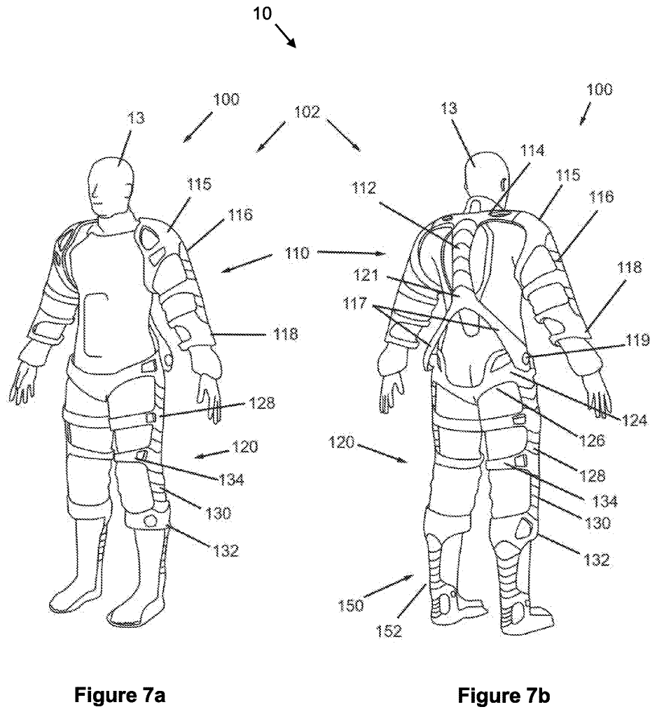

The space suit 10 also includes an outer layer 100 that is to be worn over the sensor suit 11. The outer layer 100 includes an exoskeleton 102, as shown in FIG. 7a and FIG. 7b. The outer layer 100 is sized so that the occupant 13 can fit within the exoskeleton 102. The exoskeleton 102 includes a torso section 110 that extends from the shoulders down to a hip region of the occupant 13. The torso section 110 includes a flexible spinal column 112 that in use sits adjacent an upper spinal region of the occupant 13. The flexible spinal column 112 is articulated to allow the occupant 13 to move unimpeded during movements of twisting about the spine and arcing of the spine in sideways and front to back directions. The flexible spinal column 112 is configured to support weight passing down through the flexible spinal column 112.

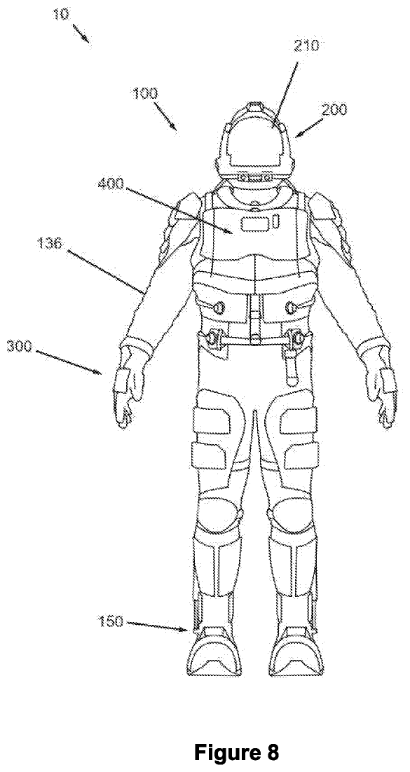

A shoulder platform 114 extends from an upper section of the flexible spinal column 112. The shoulder platform 114 extends laterally so as to be positioned over the back or rear (dorsal) side of the occupant 13. In an embodiment, the shoulder platform 114 extends laterally over a trapezius region of the occupant 13. In an embodiment, the shoulder platform 114 includes a deltoid protector 115, with arm guards 116 that terminate at a cuff 118 extending from the deltoid protector 115. The deltoid protector 115 is articulated to allow the arms of the occupant 13 to move unimpeded. The arm guards 116 are also articulated around a region of the occupant's 13 elbows to allow the arm to bend at the elbow. The cuff 118 may include a coupling mechanism that can engage with gloves 300 (see FIG. 8). The torso section 110 also has a wishbone 117 that branches out from a lower section 121 of the flexible spinal column 112. The wishbone 117 is curved to extend downwards and around the occupant 13 to connect to opposed sides of a leg section 120 so that the termini of the wishbone are positioned at a hip region of the occupant 13.

Each of the flexible spinal column 112, shoulder platform 114, deltoid protector 115, arm guards 116, and cuff 118 may be separate components that can be interchanged. This may be helpful when sizing the torso section 110 to differently sized occupants. In this way, the torso section 110 is a modular design where sub-components, such as the flexible spinal column 112, shoulder platform 114, deltoid protector 115, arm guards 116, and cuff 118, be replaced as needed.

The exoskeleton 102 also includes the leg section 120 that extends from the hip region down to a lower leg region 132 of the occupant 13. The lower leg region 132 may be positioned at an upper shin position on the occupant 13. An upper portion of the leg section has a hip portion 124. The hip portion 124 includes two leg holes 126 that can receive respective legs of the occupant 13. Extending from each side of the hip portion 124 is a respective limb portion 128. The limb portion 128 extend down to the lower leg region 132. The limb portion 128 is provided with a flexible section or articulation point 130 that corresponds with a knee region of the occupant 13. The flexible or articulated section 130 allows the occupant to bend their knee during use of the space suit 10. The limb portions 128 are also provided with leg guards 134. In the embodiment shown in FIG. 7a and FIG. 7b, the leg guards 134 in use extend circumferentially around a leg of the occupant 13. Put another way, the leg guards 134 are circular. However, in an embodiment, the leg guards 134 extend only partially around the leg of the occupant 13.

The torso section 110 and leg section 120 are pivotably connected to one another via pivot joint 119. The pivot joint 119 allows the occupant to pivot forward and backwards about the hip. The pivot joint 119 may have a limit stop to limit the range of motion, for example to prevent the occupant 13 from hyper-extension. To assist with the mobility of the occupant 13, the wishbone 117 may be formed from a resiliently deformable material. For example, the wishbone 117 may have some flexibility to allow the occupant to twist in the lower lumbar region.

The torso section 110 and leg section 120 are covered with an exoskeleton covering 136. The exoskeleton covering 136 may be formed from a plurality of layers of fabric. The types of fabric used in the exoskeleton covering 136 are typically dependent on the intended use of the protective suit system, but in the case of space suit 10 would include materials that space environments. For example, the plurality of layers of fabric may include one or more fabrics that are flame resistant, flame retardant, ballistic-proof, self-healing and/or resistant to radiation. The plurality of layers may be arranged in any sequence. However, materials that are more resistant to wear and tear are generally provided as the outer layer. The purpose of the exoskeleton covering 143 is to provide a barrier between the occupant and the outside environment. In the case of space suit 10, the exoskeleton covering 136 protects the occupant 13 from the extreme heat and cold, fast-moving micrometroids in space, and, if required, space radiation. In an embodiment, the exoskeleton covering 136 is configured to operate in a temperature ranging from −270° C. to 1,260°C.

To accommodate the movement of the occupant 13, in an embodiment the exoskeleton covering 136 includes stretchable materials, bellow-like materials and/or bellows formed in the exoskeleton covering 136 in regions that experience movement such as shoulders, elbows, and knees. As these regions are subject to more wear and tear, these stretchable materials, bellow-like materials and/or bellows formed in the exoskeleton covering 136 may be provided with a protective covering such as a replaceable wear plate.

The outer layer 100 includes a biomechanics sensor network that is configured to record data related to movement of the outer layer to monitor occupant energy usage and wear of the outer layer. For example, any movement of the limbs of the occupant 13 can be recorded by the biomechanics sensor network, such as those located in or on the exoskeleton 102, to monitor the energy expenditure of such movement. The outer layer 100 may also include sensors that monitor radiation and temperature.

The space suit 10 also has a boot 150 for each foot. As best shown in FIG. 7b, each boot 150 has a boot frame 152 that engages with the lower leg region 132. For example, the lower leg region 132 may have a coupling mechanism that can engage with an upper section of the boot frame 152. The boot locking mechanism may allow the boot 150 to be replaced as needed, such as when a larger boot is needed to accommodate larger feet of a different occupant.

The space suit 10 also has a helmet 200, as shown in FIG. 8. In use the helmet 200 connects to or engages with the outer layer 100. For example, a lower rim of the helmet 200 can lock into a helmet locking mechanism that is located on the torso section 110 and/or on the exoskeleton covering 136 at a neck region of the occupant. The helmet has a visor 210 that the occupant 13 can see through. The helmet 200 has a display that can display information associated with the space suit 10. The display may be integrated into the visor 210. The helmet 200 is also provided with a cognitive tracking sensor that can track the cognitive function of the occupant 13 in use of the space suit 10. The cognitive tracking sensor may include one or more cognitive tracking sensors.

The boot 150 can have one or more boot pressure sensors to monitor pressure being applied by the occupant 13 and/or the space suit 10 down onto a surface on which the occupant 13 is standing. The boot may also have one or more boot temperature sensors to monitor a temperature of a foot of the occupant and/or a temperature of an environment outside of the boot 150. Data generated by the boot temperature sensor and/or boot pressure sensor can be received by and stored in the data storage system. The space suit 10 also has a life support system 400. The life support system is shown in FIG. 8 generally as an external unit that sits on a chest location of the space suit 10. This location is exemplary only and the life support system 400 may be located on a back side of the space suit 10 and/or incorporated into the outer layer 100. The life support system may also include features such as sensors that are remote to the external unit. The life support system 400 is configured to provide suitable conditions for the occupant 13 to perform their duties.

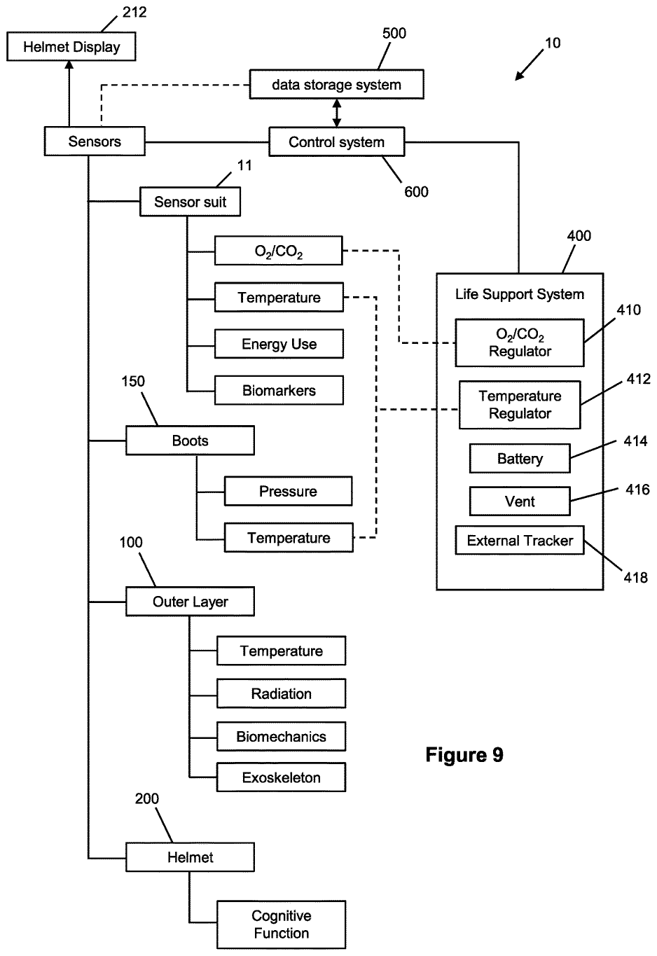

In an embodiment, the life support system 400 comprises a temperature regulator 412 (see FIG. 9) such as a temperature regulation system. In one form, the temperature regulator 412 includes an air recirculator that can recirculate heated or cooled air around an interior of space suit 10 where the occupant 13 resides. In an embodiment, the temperature regulator 412 is operable with the sensor suit 11 to regulate a temperature of the occupant 13. In such an embodiment, the temperature regulator 412 includes a thermal regulation fluid, a heat exchanger in thermal communication with the thermal regulation fluid, and a pump for pumping the thermal regulation fluid. The thermal regulator fluid can be pumped through the tubes 30 in the garment 12. For example, the fluid channels in the form of tubes 30 can have input and output lines to allow thermal regulation fluid to continually pass in and around the garment 12. The occupant 13 will typically connect the input and output lines to the temperature regulator 412 when putting the space suit 10 on.

The life support system 400 also includes an air management system, which is described as being in the form of an O₂/CO₂ regulator 410. Although the air management system is described specifically in terms of O₂ and CO₂ it can also include other gases and compounds that may be present and develop during the presence of an occupant 13 in an enclosed environment. For example, the air management system can also monitor and scrub gases such as carbon monoxide and maintain appropriate humidity levels within the space suit 10. The O₂/CO₂ regulator 410 can have a supply of oxygen, and an oxygen regeneration unit which may include a scrubber for removing carbon dioxide. The O₂/CO₂ regulator 410 functions to maintain appropriate levels of oxygen in the environment within the space suit 10 based on predefined conditions. The air management system can also include fans and/or pump to move air around an interior of the space suit 10 in which the occupant 13 would reside in use. The fans and pumps may help to evenly distribute air in the interior of the space suit 10, for example to prevent localised buildup of carbon dioxide.

The life support system 400 may also include a vent 416 that allows the environment within the space suit 10 to vent to an outside environment. For example, if a humidity level or an internal pressure exceeds a predefined threshold, the vent can open to reduce the level of humidity or internal pressure to be within the predefined threshold.

The life support system 400 may also include an external tracker 418. The purpose of the external tracker 418 is to track parameters that are external to the space suit 10. For example, external parameters may include a temperature and pressure of an external environment, the rate of change of the external temperature and pressure, a location of the space suit 10, and so on.

The life support system 400 includes a battery or battery pack that is used to power the life support system 400 and components of the space suit 10. The term “battery” includes associate componentry and functions such as a battery management interfaces and systems for managing the battery pack. The battery may be replaceable or may be fixed in or on the life support system 400 and be recharged by an external power source.

The interoperability of the various components of the space suit 10 will now be described with reference to FIG. 9. The base layer (i.e. sensor suit 11), outer layer 100, boots 150 and helmet 200 all have sensors that can monitor a range of markers and function of the occupant 13 and space suit 10. These sensors provide information that can be used by the life support system 400.

Starting with the sensor suit 11, the shoulder sensor 18, the upper arm sensor 22, the chest and heart sensor 20, the abdominal sensor 24, the upper leg sensor 26, and the knee sensor 28, all provide a stream of biomarker information about the occupant 13. These biomarkers include the occupant's O₂ levels, temperature of the occupant 13, and the amount of energy the occupant 13 is using at any given time and in what location(s). The sensor suit 11 may also include air sensors, such as O₂/CO₂ sensors to detect O₂/CO₂ levels in the environment within the space suit 10. This information generated by the various sensors of the sensor suit 11 is used by the life support system 400 to control for example the O₂/CO₂ regulator 410 and temperature regulator 412. For example, if a carbon dioxide level in the environment within the space suit 10 exceeds a predefined threshold, a scrubber or similar may be activated to reduce the concentration of carbon dioxide. Similarly, if a temperature of the occupant 13 falls outside a predefined temperature threshold, the temperature regulator 412 is activated to either heat or cool the thermal regulation fluid and then pump the heated or cooled thermal regulation fluid through the fluid channels (e.g. tubes 30) in the garment 12 to bring the temperature of the occupant 13 back within the predefined temperature threshold.

The outer layer 100 has biomechanical sensors associated with the exoskeleton 110. Movement by the occupant 13 causes the exoskeleton 110 to move, and this movement can be recorded by the biomechanical sensors. The outer layer 100 may also have sensors that can detect radiation and temperature. This information may be fed into the data storage system and/or used by the life support system 400 to respond in a pre-emptive or reactive manner to maintain the occupant within a predefined operational state (e.g. temperature, O₂ levels, and so on).

The boots 150 have temperature sensors to monitor a temperature of the feet of the occupant 13. Similar to the temperature sensors used with the sensor suit 11, if the boot temperature sensor(s) detects that a temperature of the occupants feet falls outside a predefined temperature threshold, the temperature regulator 412 is activated to either heat or cool the thermal regulation fluid and then pump the heated or cooled thermal regulation fluid through the boot 150 to bring the temperature of the occupant's feet back within the predefined temperature threshold. The predefined temperature threshold may vary depending on the location of the occupant. However, the predefined temperature threshold is correlated to maintain a temperature of the occupant with suitable physiological conditions, such as a body temperature of 37° C.

The helmet 200 is provided with sensors to monitor cognitive function. Such sensors may monitor eye movement, pupil characteristics, and voice or speech patterns of the occupant 13. If the cognitive tracking sensors detect that the cognitive function of the occupant 13 is outside predefined parameters, an alert or similar may be provided on the helmet display 212. The data generated by the sensors to monitor cognitive function may also be used by the life support system 400. For example, drowsiness detected by the sensors to monitor cognitive function may be corrected by activating the O₂/CO₂ regulator to increase an oxygen concentration inside the space suit 10.

The sensors of each space suit 10 component e.g. sensor suit 11, outer layer 100, boots 150, and helmet 200, can be controlled and operate independently of one another such that the controls for the sensor for each component are managed in a distributed manner for the space suit 10 components. The various sensors used in the sensor suit 11 may be wirelessly connected to an internet of things (IoT) network. The IoT network may also encompass medical sensors to capture biomarkers, such as those provided on sensor suit 11, along with electromechanical sensors, such as those associated with the exoskeleton 102. The battery 414 may be used to power the internet of things network and may also be used to power the control or operation of the different sensor networks of the different components of the space suit 10.

The space suit 10 also has a data storage system 500 that can store data generated by the sensors of components of the space suit such as the biomarker sensor network of the sensor suit 11 and the cognitive tracking sensors of the helmet 200. The internet of things network is connected to the data storage system 500. In use of the space suit 10, the various sensors generate data associated with various tasks. For example, movement of the occupant 13 requires contraction of the occupant's muscles which can be detected by the e.g. biomarker sensor network. The same movement can also cause the exoskeleton 102 to move, which can be detected using the biomechanics sensor network. The data generated by such movement is stored in the data storage system 500 for later analysis. For example, movement of the exoskeleton 102 can be correlated to the muscles used by the occupant 13 for said movement, and an analysis of the muscles used can help to determine if the occupant 13 is moving in the most efficient manner. Likewise, monitoring muscle activation and correlating this with movement of the exoskeleton 102 can be used to train occupants to move correctly in the space suit 10. The data stored in the data storage system 500 may be accessible in real-time through a wireless connection or can be downloaded and analysed offline. The space suit system 10 may also include a central control system 600. The central control system 600 may use information stored in the data storage system 500 and/or data generated directly by the various sensors to control the life support system 400. In this way, the control system 600 plays a role in telemetry of the space suit system 10, with the various sensors of the space suit system 10 e.g. biomarker sensor network and biomechanics sensors acting as individual telemeters. The control system 600 is shown as being a standalone component of the space suit system 10 but may be distributed throughout the components of the space suit system 10. For example, computing units associated with the sensor suit 11, the exoskeleton 102, helmet 200, and so on may collectively form the control system 600. The control system 600 may be associated with or integrated into the life support system 400. The control system 600 may also receive user inputs, such as user interface buttons on the outer covering 136 or voice commands that can be detected by the helmet 200, and process these user inputs to control the space suit system 10.

The data stored on the data storage system can be used to track and monitor the use of the space suit 10 by the occupant 13. Machine learning, artificial intelligence, predictive analytics, and so on, can be used to process the data in the data storage system. For example, if the temperature of an outside environment of the space suit 10 quickly changes, the life support system 400 can pre-emptively adjust to ensure the occupant is operating efficiently. The data stored in the data storage system 500 can also be used to construct a digital suit twin and training simulations.

In an embodiment, the space suit 10 provides a platform to help manage the human spaceflight lifecycle through energy management, low torque operational advantage, and bio-intelligence generation, resulting in astronaut performance intelligence.

In the claims that follow and in the preceding description, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the disclosure.

It is to be understood that, if any prior art publication is referred to herein, such reference does not constitute an admission that the publication forms a part of the common general knowledge in the art, in Australia or any other country.

Modifications and variations as would be apparent to a skilled addressee are deemed to be within the scope of the present disclosure.

1.-32. (canceled)

33. A protective suit system that is configured to protect an occupant in extreme environments, the system comprising:
a base layer to be worn on the occupant that is configured for thermal regulation of the occupant, the base layer including a biomarker sensor network for monitoring one or more biomarkers of the occupant;
an outer layer to be worn over the base layer, the outer layer including an exoskeleton structure that is sized so that the occupant can fit within the exoskeleton structure, and an exoskeleton covering that covers and is secured to the exoskeleton structure, wherein the outer layer includes a biomechanics sensor network that is configured to record data related to movement of the outer layer to monitor occupant energy usage and wear of the outer layer, wherein data generated by the biomechanics sensor network can be received by and stored in the data storage system; and
a data storage system that can store data generated by the biomarker sensor network.

34. A protective suit system as claimed in claim 33, wherein the biomarkers of the occupant include musculoskeletal biomarkers.

35. A protective suit system as claimed in claim 33, wherein the biomarker sensor network includes one or more biomarker sensors placed proximal to or on locations that are associated with synovial joints, heart and/or forearm of the occupant, wherein in-use the one or more biomarker sensors non-invasively monitor the one or more biomarkers of the occupant.

36. A protective suit system as claimed in claim 35, comprising a plurality of biomarker sensors being distributed on the base layer so that the one or more biomarker sensors monitor in-use one or more of the occupant's shoulders, elbows, knees, ankles, heart and forearms.

37. A protective suit system as claimed in claim 33, further comprising a life support system.

38. A protective suit system as claimed in claim 37, wherein the life support system comprises a temperature regulation system that is operable with the base layer to regulate a temperature of the occupant.

39. A protective suit system as claimed in claim 38, wherein:
temperature regulation system includes a thermal regulation fluid, a heat exchanger in thermal communication with the thermal regulation fluid, and a pump for pumping the thermal regulation fluid; and
the base layer includes a network of fluid channels through which the thermal regulation fluid can pass for regulating a temperature of the occupant.

40. A protective suit system as claimed in claim 37, wherein the life support system comprises an air management system that in use manages an air environment located within the outer layer in which the occupant is located in use of the protective suit system, the air management system configured to maintain a level of oxygen and carbon dioxide in the air environment within a predefined condition.

41. A protective suit system as claimed in claim 37, wherein the life support system comprises a battery pack for powering at least the life support system, and a battery management interface for managing the battery pack.

42. A protective suit system as claimed in claim 33, wherein the base layer includes a compression layer that is configured to apply pressure to the occupant, the compression layer being formed from stretchable material that covers an outer surface of the base layer.

43. A protective suit system as claimed in claim 42, wherein the compression layer covers an entirety of the outer surface of the base layer.

44. A protective suit system as claimed in claim 33, wherein the exoskeleton covering comprises a plurality of layers of fabric, the plurality of layers of fabric including one or more fabrics that are flame resistant, flame retardant, ballistic-proof, self-healing and/or resistant to radiation.

45. A protective suit system as claimed in claim 33, wherein the exoskeleton covering is flame retardant and ballistic-proof.

46. A protective suit system as claimed in claim 33, wherein the exoskeleton covering has stretchable bellow-like formations or materials at locations of shoulders, elbows, hips, knees and ankles of the occupant.

47. A protective suit system as claimed in claim 33, wherein the exoskeleton structure includes a torso section that is pivotally connected to a leg section, the torso section in-use extending from the shoulders down to the hips of the occupant, and the leg section in-use extending down from the hips.

48. A protective suit system as claimed in claim 47, wherein the torso section includes a flexible spinal column that branches into a wishbone at a lower section of the flexible spinal column, the wishbone is curved such that it extends down and around to attach to opposed sides of the leg section.

49. A protective suit system as claimed in claim 47, wherein the flexible spinal column also branches into a shoulder platform at an upper section of the flexible spinal column, the shoulder platform extending laterally so as to fit in-use over the back side of the shoulders of the occupant.

50. A protective suit system as claimed in claim 47, wherein the leg section includes a hip portion that extends around the hips, the hip portion including:
two leg holes that can receive respective legs of the occupant, and
a limb portion extending down each outer side of the hip portion, wherein each limb portion includes an articulation portion located at a knee region of the occupant.

51. A protective suit system as claimed in claim 33, further comprising a helmet that is connectable to the outer layer and the data storage system, wherein the helmet comprises:
a cognitive tracking sensor that can track cognitive function of the occupant; and
a display that provides a means of communicating information associated with the protective suit system to the occupant including information from the biomarker sensor network and the data storage system.

52. A protective suit system as claimed in claim 33, further comprising a boot.
